(19) United States
(12) Patent Application Publication
(10) Pub. No.: US 2004/0138097 A1
(43) Pub. Date: Jul. 15, 2004

(54) Method and treatment for treating and preventing pain associated with compression of a nerve

(76) Inventor: Bahman Guyuron, Lyndhurst, OH (US)

Correspondence Address: Amy E. Rinaldo, KOHN & ASSOCIATES, PLLC, Suite 410, 30500 Northwestern Highway, Farmington Hills, MI, 48334, US

(21) Appl. No.: 10/700,732

(22) Filed: Nov. 3, 2003

Related U.S. Application Data
(60) Provisional application No. 60/422,929, filed on Nov. 1, 2002.

(51) Int. Cl. A61K 38/16; A61N 1/00

(52) U.S. Cl. 514/2; 514/12; 607/1

(57) ABSTRACT

A method of treating pain caused by a nerve being compressed by a muscle by inactivating the muscle causing the nerve compression is provided. A treatment for eliminating pain associated with nerve compression, the treatment including a muscle inactivator for inactivating the muscle and relieving the nerve compression is also provided. An algorithm for determining the location of pain causing nerve compression is provided.

### CROSS-REFERENCE TO RELATED APPLICATIONS

This application claims the benefit of priority under 35 U.S.C. Section 119(e) of U.S. Provisional Patent Application No. 60/422,929, filed Nov. 1, 2002, which is incorporated herein by reference.

### BACKGROUND OF THE INVENTION

1. Technical Field

The present invention relates to pain management and resolution as well as prevention of the source of pain. More specifically, the present invention relates to the treatment of migraines.

2. Description of the Related Art

Migraine headaches are a very common disorder that afflicts numerous people on a regular basis. A migraine headache has been defined generally, as an episodic headache lasting a finite time, in the range of a small amount of time to days. The small amount of time could range from a few minutes to a few hours. The episodic headaches are often, but not always, associated with an aura followed by gastrointestinal discomfort, dizziness, pulsatile pain, increased pain through normal physical activity, photophobia, phonophobia, and/or visual disturbances. It is common that the discomfort and disturbance is of such a nature and frequency so as to adversely affect the afflicted individual's lifestyle.

In response to such suffering, practitioners have developed numerous treatments that have proven marginally effective if applied at the appropriate time. The treatments range from drugs to the application of various devices. None of the prior art methods effectively treat migraine headaches or eliminate the occurrence of migraine headaches. Examples of such prior art methods are disclosed in U.S. Pat. No. 5,914,129 to Mauskop, U.S. Pat. No. 5,538,959 to Mauskop, U.S. Pat. No. 4,024,279 to Zoc et al, U.S. Pat. No. 4,786,643 to Sanger et al., U.S. Pat. No. 4,916,125 to Herrling et al., U.S. Pat. No. 5,273,759 to Simmons, U.S. Pat. No. 5,639,784 to Hammarberg et al., U.S. Pat. No. 5,693,638 to Myers, U.S. Pat. No. 6,077,539 to Plachetka et al., U.S. Pat. No. 5,855,884 to Theoharides, U.S. Pat. No. 5,981,526 to Hargreaves, and U.S. Pat. No. 6,103,218 to Brucker et al. The aforementioned patents disclose the treatment of a migraine headache through the administration of a pharmaceutical compound. Such treatments require the ingestion of a variety of drugs, all of which can have negative effects in a percentage of patients.

Additionally, several methods for treating a migraine headache without the ingestion of drugs have been developed. Examples of such methods are disclosed in U.S. Pat. No. 5,795,150 to Boyd (the '150 patent), U.S. Pat. No. 5,513,656 to Boyd, Sr. (the '656 patent), U.S. Pat. No. 4,856,526 to Liss et al. (the '526 patent), U.S. Pat. No. 4,509,521 to Barry (the '521 patent), and U.S. Pat. No. 5,419,758 to Vijayam (the '758 patent). While the patents disclose treatments and preventative measures for migraine headaches, the treatment methods are neither convenient nor readily available. Further, none of the treatments have proven effective at providing permanent relief from migraine pain.

The '150 and the '656 patents both disclose treating migraine headaches by attempting to prevent its occurrence through the prevention of chronic tension. The patents disclosed devices that prevent chronic tension by the patient wearing an intra-oral device. The device is worn by a patient about the maxillary incisors such that a patient may not clench their teeth. The device is designed to be worn by a patient at all times, but is removable for eating. A limitation of such a device is that the device can become bothersome because it must always be worn. Additionally, the device is only effective in preventing migraine headaches that are caused by chronic tension and may not be effective in preventing migraine headaches caused by other factors.

The '758 patent discloses the use of an elastic band worn about the head of a patient to compress dilated blood vessels in order to provide relief of migraine headache pain. Rubber disks may be inserted between the band and the scalp to provide more localized pressure over areas with more severe pain. The wearing of such a device can become both uncomfortable and inconvenient. Additionally, the constricting of blood flow in the areas about the head may be dangerous if used by a patient without medical supervision.

The '521 and the '526 patents both disclose migraine and headache relief apparatuses that relieve pain through the application of electric pulses via the apparatuses. The application of electric pulses to an area about the cranium of a patient may be dangerous unless performed under the supervision of a doctor and such treatment may be too invasive for some patients.

Other methods of treating migraine headaches and related maladies have concentrated on the pulsating of a laser light to the afflicted areas. Such examples are disclosed in U.S. Pat. No. 5,514,168 to Friedman (the '168 patent) and U.S. Pat. No. 5,640,978 to Wong (the '978 patent). Both of the patents may not be entirely effective and require the availability of a laser light system for use.

The '168 patent discloses the application of a low power laser light to an intra-oral zone of tenderness often encountered in migraine headaches. However, not all migraine headaches produce an intra-oral zone of tenderness and thus could not be treated using the method of the '168 patent.

The '978 patent broadly discloses the treatment of a variety of muscular pains. The treatment includes a probe that is placed in close proximity to a pain, such as a muscular pain from a migraine headache, and low pulses of a laser are transmitted to the muscle via the probe. The method requires the availability of a laser system and may be too invasive for some patients.

It would therefore be useful to develop a treatment of migraine headaches that does not have negative side effects and provides permanent relief from migraine pain.

### SUMMARY OF THE INVENTION

According to the present invention, there is provided a method of treating pain caused by a nerve being compressed by a muscle by inactivating the muscle causing the nerve compression is provided. A treatment for eliminating pain associated with nerve compression, the treatment including a muscle inactivator for inactivating the muscle and relieving the nerve compression is also provided. An algorithm for determining the location of pain causing nerve compression is provided.

### BRIEF DESCRIPTION OF THE DRAWINGS

Other advantages of the present invention are readily appreciated as the same becomes better understood by reference to the following detailed description when considered in connection with the accompanying drawings wherein:

FIG. 1 is an intraoperative photograph documenting penetration of the corrugator supercilii muscle by the branches of the supraorbital nerve;

FIGS. 2A and B are photographs showing preoperative (FIG. 2A) and postoperative (FIG. 2B) views of a patient with migraine headaches, revealing the magnitude of corrugator supercilii muscle hypertrophy while frowning, while attempting to frown after removal of corrugator supercilii muscles, transection of the zygomaticotemporal branch of the trigeminal nerve, and temple soft-tissue repositioning; and

FIG. 3 is a flow chart depicting the flow of information for the algorithm of the present invention.

### DETAILED DESCRIPTION OF THE INVENTION

Generally, the present invention provides a treatment that both treats and prevents the occurrence of pain associated with the compression of a nerve.

By “pain associated with the compression of a nerve” it is intended to include any pain that is caused by the compression of a nerve by muscle tissue.

The term “patient” means and refers to an individual afflicted with a pain associated with the compression of a nerve, such as a migraine headache. The term “migraine headache” means and refers to episodic headaches that are often, but not always, associated with gastrointestinal discomfort, dizziness, pulsatile pain, increased pain through normal physical activity, photophobia, phonophobia and/or visual disturbances.

Compression can be caused by muscle contractions about a nerve that passes through the muscle tissue. The contraction is usually involuntary and can be caused by various sources. Some sources are well known while others have yet to be elucidated. Examples of muscles that compress nerves include, but are not limited to, corrugator supercilii, occipitalis, and temporalis.

Nerves that are compressed and therefore treated in accordance with the present invention can be either sensory or motor nerves. The treatment of the present invention restores function to the compressed nerve by deadening the muscle that is compressing the nerve. The treatment can selectively restore functionality. In other words, the physician can select the specific site for treatment and at the site for treatment, the physician can elect to treat the motor and/or sensory nerves.

The present invention provides a method and treatment for treating migraine and other pain associated with the compression of nerves that travel through muscle. The treatment of the present invention targets nerves that are compressed by muscle such that upon activation of the muscle, the muscle contracts or clenches, thereby compressing the nerve, which, in turn, causes pain. The pain can occur anywhere within a body, as long as the pain is related to the compression of a nerve by muscle tissue. Examples of such pain can include, but are not limited to, migraine, back pain, and other similar pain.

More specifically, the present invention provides a method of functionally ablating (i.e. paralyzing or denervating) the muscle surrounding the nerve being compressed, thereby eliminating compression and the resulting pain caused by the muscle compression about a nerve. Inactivation or ablation of the muscle can be attained through removing, deadening, numbing, or otherwise incapacitating the muscle such that the muscle is incapable of contraction. Treatment can be effectuated by surgical removal of the muscle, injection of a deadening or an otherwise permanently disabling composition(s), or by any other means presently available to effectuate inactivation of the muscle. Examples of surgical treatments include, but are not limited to, the use of laser energy for ablating the muscle, surgical resection of the muscle, use of radio frequencies to ablate the muscle, and other such methods as are known to those of skill in the art to effectuate the same result.

Muscle inactivation can be also effectuated using a compound, such as a pharmaceutical, which can be administered systematically or directly into muscle. The purpose of the administration of the compound is inactivation of a specific muscle that is compressing the sensory (pain sensing) nerve in question. One example of such a compound is Botulinum toxin (hereinafter “Botox”). However, the present invention is not limited strictly to the Botox compound but can include any other compounds can inactivate muscle without any deleterious side effects. The Botox can be selected from the group consisting of Botulinum toxin types A, B, C, D, E, F, and G. Any serotype of Botox can be used in the treatment method described herein. As further elaborated in the examples, Botox has effectively been used to at least temporarily deaden the muscle surrounding a nerve. For example, Botox was successfully administered to the corrugator supercilii muscle of patients presenting with migraine pain to permanently alleviate migraine pain.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve deadening including, but not limited to, elimination or lessening of symptoms and other indicators as are selected as appropriate measures by individuals skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered subcutaneously or intramuscularly. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such as cottonseed oil, sesame oil, olive oil, soybean oil, corn oil, sunflower oil, or peanut oil and esters, such as isopropyl myristate, may also be used as solvent systems for compound compositions. Additionally, various additives that enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to individuals skilled in the art.

The quantity to be administered will vary for the patient being treated and will vary from about 100 ng/kg of body weight to 100 mg/kg of body weight per day and preferably will be from 10 mg/kg to 10 mg/kg per day.

Critical to the treatment is pinpointing the source of the pain; that is, the specific muscle or muscles that are compressing the pain causing nerve or nerves. The present invention provides a site-specific treatment resulting in alleviation of the source of the pain, as opposed to a general anesthetic or nerve deadening effect. Thus, a general systemic sensory deadening is not required. Rather, site-specific treatment through functional ablation is performed only on the muscle directly causing pain.

The present invention also provides a protocol for determining the trigger points of pain associated with the compression of a nerve shown in FIG. 3. Preferably, the protocol is automated such that a patient's symptoms and indicia resulting from a physician's examination are entered into a program containing the protocol. The program then provides the physician with a diagnosis of the location or locations of nerve compression and therefore location(s) for treatment. The protocol includes an algorithm that converts the symptoms and indicia into a specific diagnosis, thereby enabling a physician to more specifically treat the cause of the pain.

The invention is further described in detail by reference to the following experimental examples. The examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

A study was conducted to investigate the role of removal of corrugator supercilii muscles, transection of the zygomaticotemporal branch of the trigeminal nerve, and temple soft-tissue repositioning in the treatment of migraine headaches. Using the criteria set forth by the International Headache Society, a neurologist evaluated patients with moderate to severe migraine headaches, to confirm a diagnosis. Subsequently, the patients completed a comprehensive migraine headache questionnaire and a plastic surgeon injected 25 units of botulinum toxin type A (Botox) into each corrugator supercilii muscle. The patients were asked to maintain an accurate diary of their migraine headaches and to complete a monthly questionnaire documenting pertinent information related to their headaches. Patients in whom the injection of Botox resulted in complete elimination of the migraine headaches then underwent resection of the corrugator supercilii muscles. Patients who experienced only significant improvement underwent transection of the zygomaticotemporal branch of the trigeminal nerve with repositioning of the temple soft tissues, in addition to removal of the corrugator supercilii muscles.

Once again, patients kept a detailed postoperative record of their headaches. Of the 29 patients included in the study, 24 were women and five were men, with an average age of 44.9 years (range, 24 to 63 years). Twenty-four of 29 patients (82.8 percent, p<0.001) reported a positive response to the injection of Botox, sixteen (55.2 percent, p<0.001) observed complete elimination, eight (27.6 percent, p<0.04) experienced significant improvement (at least 50 percent reduction in intensity or severity), and five (17.2 percent, not significant) did not notice a change in their migraine headaches. Twenty-two of the 24 patients who had a favorable response to the injection of Botox underwent surgery, and 21 (95.5 percent, p<0.001) observed a postoperative improvement. Ten patients (45.5 percent, p<0˜01) reported elimination of migraine headaches and 11 patients (50.0 percent, p<0.004) noted a considerable improvement. For the entire surgical group, the average intensity of the migraine headaches reduced from 8.9 to 4.1 on an analogue scale of 1 to 10, and the frequency of migraine headaches changed from an average of 5.2 per month to an average of 0.8 per month. For the group who only experienced an improvement, the intensity fell from 9.0 to 7.5 and the frequency was reduced from 5.6 to 1.0 per month. Only one patient (4.5 percent, not significant) did not notice any change.

The follow-up ranged from 222 to 494 days, the average being 347 days. In conclusion, the study confirms the value of surgical treatment of migraine headaches, inasmuch as 21 of 22 patients benefited significantly from the surgery. It is also evident that injection of Botox is an extremely reliable predictor of surgical outcome. (Plast. Reconstr. Surg. 109: 2183, 2002.)

### Example 2

There was recently reported the unexpected elimination or improvement in migraine headaches following rejuvenation of the forehead involving removal of hyperactive corrugator supercilii muscles. A retrospective study indicated that of 9 patients with migraine headaches who underwent a forehead aesthetic procedure, 15 (38.5 percent) reported complete disappearance of their headaches and 16 patients (41. percent) observed significant improvement, within a mean follow-up period of 46.5 months.

A prospective study was conducted to confirm the findings of the retrospective study. The patients likely to have a successful outcome following surgery were identified using injection of botulinum toxin type A (Botox, Allergan, Inc., Irvine, Calif.). The patients underwent surgical removal of the corrugator supercilii muscles alone or in conjunction with transection of the zygomaticotemporal branch of the trigeminal nerve and repositioning of the temple soft tissues to prevent recoaptation of the nerve.

Patients and Methods

Patients who complained of migraine headaches were initially evaluated by a neurologist to confirm the diagnosis of migraine headaches on the basis of criteria set forth by the International Headache Society. The patients completed a comprehensive migraine headaches questionnaire that contained 56 items and recorded their symptoms during the month before the procedures. Frequency, duration, characteristics, and severity of headaches, graded on an analogue scale from 1 to 10 (10 being very severe) were documented. Corrugator supercilii muscle hypertrophy was clinically assessed in all patients and graded from 1 to 5 (5 being significant). Patients with medical or neurologic conditions likely to induce migraine headaches were considered ineligible for the study. Similarly, patients who were deemed unacceptable surgical risks and patients who were pregnant or nursing at the time of neurologic examinations were excluded from the study. In addition, patients whose migraine headaches responded to over-the-counter medications were barred from the study. Patients who were in good health, between 18 and 75 years old, and who experienced two or more moderate to severe migraine headaches each month were considered eligible for the study.

Twenty-five units of Botox were injected by the plastic surgeon into each corrugator supercilii muscle of the volunteers who fulfilled the study criteria. After the injection of Botox, patients were asked to refrain from using any regular prophylactic migraine headaches medications and to keep an accurate diary of any headaches, including the details of symptoms, location, severity, and frequency of the headaches. The patients were instructed, however, to use their migraine headache medications if they experienced an acute attack.

The patients who responded favorably to the injection of Botox with complete elimination of migraine headaches for at least six weeks, or the patients who observed significant (at least 50 percent) reduction in intensity or severity of their migraine headaches, were considered suitable candidates for surgery. Subjects who observed complete elimination of their migraine headaches following Botox injection underwent a thorough resection of each corrugator supercilii muscle. The patients who noted only improvement following Botox injection underwent a combination procedure similar to an endoscopic forehead rejuvenation. The treatment consisted of complete removal of each corrugator supercilii muscle, transection of the zygomaticotemporal branch of the trigeminal nerve, and repositioning of the temple soft tissues to minimize the potential for recoaptation of the transected nerves.

After injection or surgery, patients kept a diary of their headaches and completed a questionnaire on a monthly basis. A reduction in intensity or frequency of at least 50 percent was considered improvement. The results were then statistically analyzed using binomial distribution Z statistics with continuity correction. The p values were calculated by comparing the observed proportion on the basis of 29 patients undergoing Botox injection against 1 percent improvement and the proportion on the basis of 22 patients who underwent surgery when compared against 5 percent improvement, respectively. When comparing the pre-surgical and post-surgical frequency and intensity data, the two-tailed Wilcox was used on signed rank test.

Surgical Techniques

For the transpalpebral approach, a skin incision was made in the upper tarsal crease of each eyelid, approximately 1 inch in length, and deepened through the orbicularis muscle only. In the anatomic plane between the orbicularis muscle and orbital septum, the dissection was continued cephalad until the corrugator supercilii muscles were exposed. Preserving the supraorbital and supratrochlear nerves, both corrugator supercilii muscles were removed as thoroughly as possible. A small amount of fat, often protruding on the medial aspect of the upper eyelid, was harvested through a small opening in the orbital septum and applied to the corrugator supercilii muscle site. The fat graft was sutured in place using 6-0 polyglactin (Vicryl). The skin incision was repaired using 6-0 plain catgut.

The combination of corrugator supercilii muscle resection, temporal release, and transection of the zygomaticotemporal branch of the trigeminal nerve was performed through an endoscopic approach. Five half-inch incisions were made, one in the center and two on either side in the temple area. The endoscopic access devices were inserted. A subperiosteal dissection was carried to the supraorbital rim, lateral orbital rim, zygomatic arch, and malar region. The zygomaticotemporal branch of the trigeminal nerve was transected and coagulated. The periosteum and the arcus marginalis were released over the lateral orbital region and the supraorbital area to allow repositioning of the tissues. The supraorbital nerves and each corrugator supercilii muscle were exposed. The glabellar area was dissected and the periosteum was released. Next, each corrugator supercilii muscle was removed as completely as was feasible. Fat graft, harvested from the temporal region, deep to the intermediate or deep temporal fascia, was applied to the corrugator supercilii muscle site. Fixation was achieved with 3-0 polydioxanone fascia sutures laterally and bone tunneling through the medial incision in the temple area. A suction drain was placed in position and anchored to the skin using 5-0 plain catgut. The incisions were repaired using a combination of 5-0 polyglactin (Vicryl) and 5-0 plain catgut.

Results

Twenty-nine patients with a confirmed diagnosis of moderate to severe migraine headaches constituted the study group. There were 24 women and five men, closely matching the national gender-based distribution of the migraine headache patient population (Table I). The patients ranged in age from 24 to 63 years, with an average age of 44.9 years. The average corrugator supercilii muscle hypertrophy was 4.7, ranging from 4 to 5.

Outcome Following Injection of Botox

After injection of Botox, 24 of the 29 patients (82.8 percent, p<0.001) noted an improvement in their migraine headaches. In 16 patients (55.2 percent, p<0.001), migraine headaches disappeared completely (Table II), whereas eight patients (27.6 percent, p<0.04) observed significant improvement for six consecutive weeks or more. The average frequency decreased from 6.4 to 2.1 per month and the intensity fell from 8.6 to 6.1 (Table II). Five patients (17.2 percent, not significant) reported no change (Table II). Two patients (6.9 percent) noted transient unilateral upper eyelid ptosis that lasted two weeks for one patient and three weeks for the other patient.

Outcome Following Surgery

Of the 24 patients who noted a favorable response to injection of Botox, 22 underwent surgery. The group included 18 women and four men ranging in age from 24 to 58 years old. Of the 22 patients who underwent surgery, 21 (95.5 “percent, p<0.001) observed an improvement in the migraine headaches. Ten patients (45.5 percent, p<0.01) noted elimination of migraine headaches (Table II) and 11 (50.0 percent, p<0.004) noted a significant improvement (Table II). The average intensity of migraine headaches for the entire surgical group was reduced from 8.9 to 4.1 (p<0.04) on an analogue scale of 1 to 10, and the frequency changed from 5.2 to an average of 0.8 (p<0.001) per month. When the group with improvement only was analyzed, the intensity fell from 9.0 to 7.5 and the frequency changed from 5.6 to 1,0 migraine headaches per month. Only one patient failed to notice an improvement (Table II). The follow-up ranged from 222 to 494 days, with an average of 347 days.

There was no incidence of wound infection. Three patients received an infusion of desmopressin (DDA VP) for moderately excessive bleeding during surgery. All patients experienced some numbness in the temple area lasting 1 to 6 months, with an average of 2.3 months. All patients reported complete sensory recovery during the follow-up period. All patients noted aesthetic improvement, with the disappearance or diminution of the forehead line and better eyebrow position. The patient who failed to notice enough improvement stated that the location and the pattern of her migraine headaches were different when compared with her preoperative headaches. Her medical history confirmed long-standing perinasal sinus disease, and an internal nose examination revealed a notable deviation of the septum. Similarly, three of eleven patients who reported only improvement in migraine headaches stated that the pattern and the location of the migraine headaches were different postoperatively.

Discussion

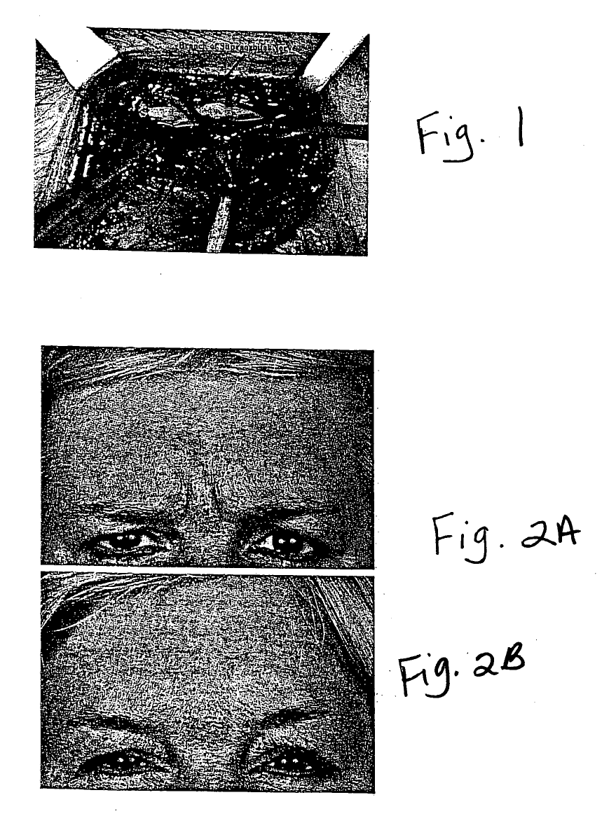

The surgical approach involves the peripheral branches of the trigeminal nerve and muscles affecting the terminal branches; as a result, the surgical procedures are less complicated and serious morbidity is extremely rare. The role of the trigeminal nerve in the pathogenesis of migraine headaches has been studied for over 30 years. It is postulated that stimulation of the nerve results in release of neuropeptides such as substance P, calcitonin gene-related peptide, and neurokinin A. The peptides cause neurogenic inflammation. What activates the terminal branches of the trigeminal nerve, however, was previously unknown. The nerves are stimulated by strong contraction of the corrugator supercilii and the temporalis muscles. The supratrochlear and supraorbital nerves pierce the corrugator muscle to reach the cutaneous level (FIG. 1). Whereas the main trunk of the supratrochlear nerve passes through the corrugator supercilii muscles, only branches of the supraorbital nerve, rather than the main nerve, traverse in between muscle fibers. The zygomatcotemporal branch of the maxillary division exits from the orbit, wraps around the lateral orbital wall, and exits from the temporalis fascia and muscle to reach the cutaneous level. The nerve can be irritated by being compressed between the temporalis muscle fibers or by being pressed against the lateral orbital wall by the muscle. The occipitalis muscle, by compressing the greater occipital nerve, can also result in head pain and possibly migraine headaches. Finally, perinasal sinus linings (frontal, ethmoid, maxillary, and sphenoid) can serve as a trigger point, if irritated by abnormal air turbulence. Turbulence can result from septal deviation or abnormal sinus drainage caused by enlargement of the turbinates. The ophthalmic and maxillary divisions of the trigeminal nerve innervate the cavities. The patient who did not benefit from the surgery can have suffered from activation of another dormant trigger point that was located in the sinus cavities. Identification and elimination of the sinus trigger points results in a significant improvement in migraine headaches.

The patients in the study who did not respond completely to the removal of corrugator supercilii muscles or transection of the zygomaticotemporal branch of the trigeminal nerve harbor other trigger points that can be identified. An algorithm has been developed to detect trigger points in a sequential fashion. Patients with migraine headaches receive an injection of Botox in different trigger sites, in a logical and stepwise manner. After the trigger points are identified using the comprehensive algorithm, the trigger points are eliminated with surgical maneuvers. Patients who undergo surgery are then compared with the patients who receive placebo injection and serve as a control group, selected on a random basis.

The statistical analysis for the present study was conducted assuming spontaneous disappearance of migraine headaches in one percent of the patients in the group who underwent injection of Botox and five percent of the patients who were subjected to surgical amelioration of the migraine headaches.

In the prospective study, careful patient selection and injection of Botox enabled us to identify the patients who can have a high likelihood of benefiting from removal of the corrugator supercilii muscles. Use of Botox has proved to be an extremely reliable prognosticator. Botox is currently being studied for temporary treatment and prevention of migraine headaches with or without aura. Injection of Botox into the frontalis, temporalis, glabellar, and occipital areas has been found to reduce the severity and frequency of migraine headaches in small studies. Botox can also be as effective in treating migraine headaches as it is in treating tension-type headaches. In addition, two pilot open-labeled studies have shown improvement in headache severity scores and reduction in headache duration in patients with chronic and episodic tension-type headaches.

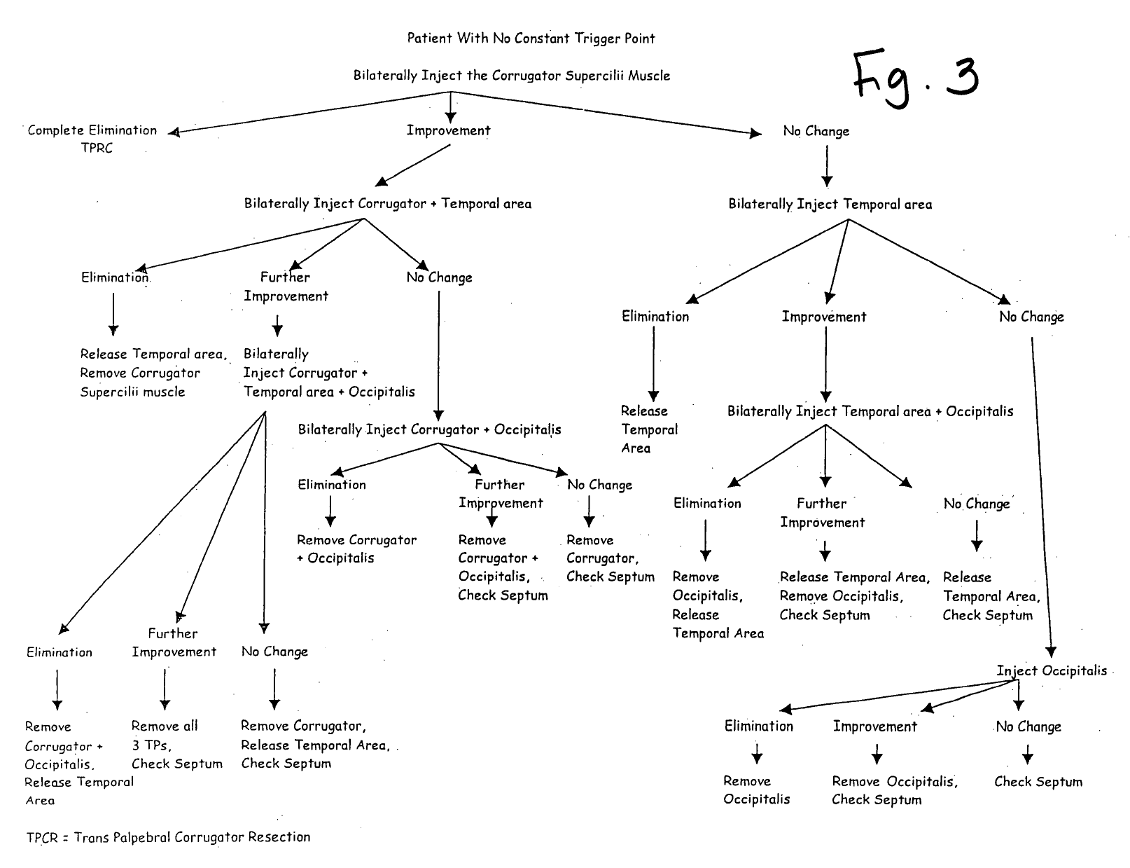

Botox inhibits release of acetylcholine at the neuromuscular junction, thereby decreasing muscle tone. The majority of non-organic headaches are related to irritation of the trigeminal nerve branches, resulting in inflammation and release of neuropeptides. When the inflammation reaches the meninges, it results in localized inflammation, inducing severe headaches, nausea, photophobia, and other characteristics of migraine head-aches. Considering that a vast number of migraine headaches are provoked by stress or light exposure and many of the patients exhibit significant hypertrophy of corrugator muscles (FIG. 2), the muscle is a cardinal factor and impingement of the trigeminal nerve branches becomes more compelling. Botox, like surgical ablation, by virtue of paralyzing the offending muscle, eliminates the trigger point, hence avoiding the migraine headaches.

Two of the five patients who did not respond to the injection of Botox in the corrugator supercilii muscles elected to undergo forehead rejuvenation. Both patients reported complete elimination of migraine headaches after surgery. Because transection of the zygomaticotemporal branch of the trigeminal nerve was part of the procedure, it was performed on the patients who did not observe elimination of the migraine headaches after injection of Botox in the corrugator supercilii muscles. The nerve is another trigger point, being compressed by the temporalis muscle.

In conclusion, facial muscles play an important role in inducing migraine headaches and elimination of the impinging effects on the peripheral branches of the trigeminal nerve has a prodigious role in the treatment of migraine headaches. Because 21 of 22 patients responded positively to surgery, it was concluded that surgical treatment of migraine headaches is successful. Even in patients who did not experience complete elimination of migraine headaches, the reduction in frequency was remarkable. Considering that 21 of 22 patients (95.5 percent, p<0.001) who were selected to undergo surgery on the basis of favorable response to Botox injection enjoyed a positive outcome from the surgery, it is logical to conclude that Botox is an extremely reliable prognosticator of the surgical outcome for the treatment of migraine headaches.

### Example 3

The objectives of the project were to identify the trigger points of subjects with moderate to severe migraine headaches (MH) and deactivate the sites surgically. Over 50 percent of the approximately 26,000,000 Americans with MH can benefit from the treatment in accordance with the present invention. Initially, diagnosis of MH was confirmed by neurologists and the nasal septum was examined by the surgeon. All patients were asked to complete health-related, SF-36, and Migraine Disability Assessment (MIDAS) questionnaires before any treatment. One hundred patients, chosen at random, underwent injection of botulinum toxin (Botox) using an algorithm of the present invention. Another 100 patients underwent injection of 0.5 cc of saline as a placebo and served as a control group for the first year. If the injection of Botox identified one or several trigger points evidenced by complete elimination or significant improvement (50% reduction in severity or frequency) of the MH over a period of at least six consecutive weeks, the patient was considered a candidate for surgery. In an unlikely case that injection of Botox following the algorithm fails to result in complete elimination of MH and the patient exhibits sufficient septal deviation, septoplasty can be recommended to the patient. The 100 patients that serve as the control for the first year can have the option of undergoing Botox injection, detection of trigger points, and surgical deactivation of the trigger points in the second year. Surgery can include removal of the corrugator supercilii muscle, release of the temple area with transection of the zygomaticotemporal branch of the trigeminal nerve and temple repositioning, removal of the occipitalis muscle, and/or septoplasty, singly or in combination, depending on the response to the Botox injection and the septal pathology. All operations can be conducted as an outpatient procedure under sedation, except for septoplasty, which can be done under general anesthesia. All patients kept a headache diary and completed the SF-36, MIDAS, and migraine specific quality of life (MSQ) questionnaires one year and five years after surgery. The control group also completed the questionnaires after one year and five years of follow-up and report details of medical expenses related to their MH. The results can be statistically analyzed for surgical outcomes, quality of life, and economic difference between the control group and subjects that undergo Botox injection and surgical elimination of the trigger points, after the first year.

After the diagnosis of migraine headaches was confirmed and the patient was deemed suitable for inclusion in the study, patients were seen at a plastic surgery clinic. The patients completed all of the questionnaires and underwent injection of Botox. Patients were followed closely until the trigger points were identified and the suitable surgical procedure has been selected.

The project identified the trigger points for subjects with migraine headaches (MH), deactivated the trigger sites surgically, verified the results of the previous retrospective and pilot studies concerning the surgical treatment of MH in a larger scale over a long follow-up period, and to conducted a comprehensive outcome study.

The preliminary results of an ongoing prospective pilot study confirmed the findings from the retrospective study, that removal of the corrugator supercilii muscle (CSM) can eliminate or improve MH in approximately 80% of patients. The pilot study also has identified the corrugator supercilii muscle as the most common trigger site impinging the supratrochlear and/or supraorbital nerve and the temporal is muscle as the second most common trigger site compressing the zygomaticotemporal branch of the trigeminal nerve. There are, however, other less common trigger points, such as the occipital nerve and nasal sinuses.

Most nonorganic MH are caused by either a single or multiple trigger points, largely within the distribution of the trigeminal nerve branches, which are compressed by the surrounding muscles such as the corrugator and temporalis muscle. In a majority of patients, the trigger point is the CSM, irritating the supratrochlear and/or supraorbital branches of the trigeminal nerve, which pierce the muscle to reach the cutaneous level. In the temporal region the zygomaticotemporal branch of the maxillary division is compressed between the muscle and the zygomatic bone or within the temporalis muscle. Additionally, in the occipital region, the greater occipitalis nerve can be impinged by the occipitalis muscle, on rare occasions. Infrequently, branches of the trigeminal nerve in the sinus mucosa can act as a trigger point, as a result of deviated nasal septum and abnormal sinus physiology causing inflammation of the sinus lining, a known phenomenon that frequently results in non-migraine headache as well.

Deactivating the trigger points can be accomplished by removal of the corrugator muscle, dissection of the temporal area and release and transection of the zygomaticotemporal branch of the maxillary division, and removal of occipitalis muscle, singly or in a variety of combinations depending upon the patient response to the injection of Botox. The muscles are expendable and the temporal release does not result in a significant functional loss. Transection of the zygomaticotemporal branch of the trigeminal nerve, which is routinely performed during extensive craniofacial surgery, can only result in transient anesthesia of the temple area. Even permanent anesthesia or paresthesia in the region can seldom be of any significant disturbance to the patient. A permanent neurological deficit from transection of the nerve can be extremely unlikely. Furthermore, septoplasty on a patient who has sufficient pathology can revert the abnormal airflow into the sinuses, thus resulting in abolishment of MH, in addition to improving breathing in most patients.

The method of the present invention includes first to identify trigger point(s) on each patient by injecting botulinum toxin (BOTOX) to CSM, temporalis muscle, and/or occipitalis muscle following the specific protocol disclosed herein. Furthermore, the surgical removal of CSM, release of temporal area, removal of the occipitalis muscle, or septoplasty (along with inferior turbinectomy, if necessary), singly or concurrently, depending on the response to Botox, can eliminate the identified trigger points.

Retrospective Study

Prompted by the above findings, charts of the patients who had undergone any type of forehead rejuvenation that included removal of the CSM were reviewed and pertinent data was collected. An initial simple questionnaire was sent to the patients inquiring whether they had MH prior to surgery and whether the headaches had disappeared after the forehead rejuvenation procedure. Having received sufficient affirmative answers, a more elaborate questionnaire was-then designed and the patients were contacted either through the questionnaire alone, or in combination with a phone interview and office visits. Every patient who responded positively for having MH preoperatively was personally interviewed. The data was analyzed to assure that the MH diagnosis was based on the criteria set forth by the International Headache Society for the diagnosis or MH.

Retrospective Study Results

Of the 314 patients, 265 were available for follow up. Thirty-nine (15.7%) of who had migraine headaches that fulfilled the International Headache Society criteria. Thirty-one of the 39 (79.5%) with preoperative migraine noted elimination or improvement in migraine headaches immediately after surgery (p<O.OOOI; by McNemar test), and the benefits lasted over a mean follow-up period of 47 months.

Study Results

Of the 29 patients who received Botox injection in the CSM, 16 (55%) experienced complete elimination of MH, 7 (24%) observed significant improvement for at least 6 weeks, and 6 (21%) had some or no response. The patients who noted complete disappearance of MH after the injection of Botox underwent surgical elimination of the CSM only.

Patients who experienced, partial but significant response to the Botox injection, underwent removal of CSM as well as temporal release with transection of the zygomaticotemporal nerve, a routine procedure performed during endoscopic forehead rejuvenation. The latter maneuver was added to the procedure based on the observation that several patients, including two of the patients who did not respond to the injection of Botox in the study, had experienced complete elimination of MH following an endoscopic forehead rejuvenation when the sole objective was aesthetic improvement. The finding, along with the success of Botox when injected in the temple area on the patients who were nonresponsive to the injection of Botox in the CSM alone, lead the identification of the zygomaticotemporal nerve as the second trigger point. Of the 21 patients (18 women and 3 men) that have undergone surgery to date, the preliminary review of the results has been extremely encouraging and confirms the findings of the retrospective study. With an average follow-up of 132 days, 16 patients have experienced complete elimination of their MH, 4 have noted a significant decrease in intensity and/or duration of their MH, and 1 has had no change in MH symptoms. Prior to surgery the 21 patients had experienced an average of 5.3 MH per month with an average severity of 8.9 (on a scale of 1 to 10, with 10 being the most severe). Of the 4 patients who experienced an improvement in their MH symptoms following surgery the average intensity was reduced from 8.9 to 5.7 and the frequency was reduced from 5.3 to 1.25 per month.

The positive response from the study is higher compared to the retrospective study due to the fact that some of the patients with improved or eliminated MH in the retrospective study were excluded due to the somewhat unjustified rigorous criteria that were exercised. However, the follow assess the long-term effect of the procedures.

Corrugator Supercilii Muscle Resection and Migraine Headaches

The study was conducted by the patients to determine whether there is an association between removal of the CSM and elimination, or significant reduction, of MH. Questionnaires were sent to 314 consecutive patients who had undergone CSM resection during endoscopic, transpalpebral, or open forehead rejuvenation procedures. The patients were queried as to whether they had a history of MH and, if so, whether the headaches significantly improved or disappeared following surgery. If the answer was affirmative, then the: patients were further questioned about the duration of the improvement or cessation of headaches and the relationship to the timing of the surgery. After initial evaluation of the completed questionnaires, a telephone interview was conducted to confirm the initial answers and to obtain further information necessary to ensure that the patients had a proper diagnosis based on the International Headache Society criteria for MH. The charts of the patients who had MH were studied to ascertain and classify the type of surgery they had undergone. Patient demographics were reviewed, and the results were statistically analyzed.

Of the 314 patients, 265 (84.4%) responded. Of the group, 16 patients were excluded because of the provision of insufficient information to meet the International Headache Society criteria, the presence of organic problems, and other exclusions mandated by study design. Thirty-nine (15.7%) of the remaining 249 patients had MH that fulfilled the Society criteria. Thirty-one of the 39 (79.5%) with preoperative migraine noted elimination or significant improvement in MH immediately after surgery (p<O.OOO 1; by McNemar test), and the benefits lasted over a mean follow-up period of 47 months. When the respondents with a positive history: of MH were further divided, 16 patients (p<O.OOO 1; by McNemar test) noticed improvement over a mean follow-up period of 47 months, and 15 (p<O.OOO 1; by McNemar test) experienced total elimination of their MH over a mean follow-up period of 46.5 months. When divided by MH type, 29 patients (74%) had nonaura MH. Of the patients, the headaches disappeared in 11 patients, improved in 13 patients, and did not change in five patients (p<O.OOOI). Ten patients experienced aura-type headaches, which disappeared or improved in seven of the patients and did not change in three of the patients (p<0.000 1). The study proves for the first time that there is indeed a strong correlation between the removal of the CSM and the elimination, or significant improvement, of MH. (Plast. Reconstr. Surg. 106:429, 2000.)

A prospective study was conducted of 29 patients diagnosed by the neurologist of the research team as having MH. The group had the CSM injected with Botox initially as a prognostic indicator. Of the 29 patients who received Botox injection, 16 (55%) noted complete elimination of MH for at least 6 weeks, 7 (24%) observed significant improvements, and 6 (21%) had no response. Patients who had complete response to the Botox underwent surgical elimination of the CSM only. Patients who noted partial but significant response to the Botox injection, underwent a corrugator supercilii removal as well as temporal release and transection of the zygomaticotemporal branch of the frontal nerve. Of the 21 patients operated on to date, the preliminary review of the results confirms the findings of the retrospective study. In fact, the positive response is higher due to the fact that some of the patients with improved or eliminated MH in the retrospective study were excluded due to stringent criteria for inclusion.

Aesthetic Indications for Botulinum Toxin Injection

In 1993, the effects of commercially available botulinum toxin was evaluated on 14 hyperactive corrugator muscles, 14 procerus muscles, one case of congenital aplasia of the depressor labii inferioris muscle, and one case of introgenic injury to the ramus mandibularis branch of the facial nerve with paralysis of the depressor labii and mentalis muscles. Of the 31 muscles injected, 28 were appropriately paralyzed with the initial injection. The desired results were obtained in the 3 remaining muscles following a second injection. The ability to frown was nullified in all subjects, resulting in the elimination of glabellar lines. Facial symmetry was achieved in both patients with muscle imbalance. The average duration of the paralysis was 8 weeks, with a range of 2 to 16 weeks. However, the period was prolonged in the latter part of the study with an adjustment of the toxin dose.

The results demonstrate that botulinum toxin injected into overactive facial muscles does produce a predictable and reversible paralysis and eliminates or ameliorates deep frown lines.

Botulinum Toxin Type A (BTX-A) for Migraine

Migraine is an episodic disorder with neurologic, gastrointestinal and autonomic symptoms. Over 17% of women and 6% of men suffer from migraine, acute and prophylactic therapies are ineffective for many and a long-acting, well-tolerated, prophylactic therapy is needed. Local injections of BTX-A have been used safely for dystonia, spasticity, tremor, and other disorders of inappropriate muscular contraction, and in limited patients with tension headache. Chronic migraine patients were identified through movement disorder/dystonia and cosmetic surgery clinics. BTX-A as Botox was injected into glabellar, temporalis, corrugator and occiptial muscles. Response was scored as complete (elimination of headaches); partial improvement (at least 50% reduction in frequency or severity of headaches) and non-responders (less than 50% reduction in frequency or severity of headaches, or lost to follow-up). Follow-up exceeded one year. Ninety-six patients (average age 41.6:i: 9.0 years) were treated: 49 (51%) had complete improvement; 27 (28%) had partial improvement; 20 (21%) had no response. Average dose was 25.7±14.2 units. Benefit persisted 3.6±2.4 months for complete responders and 2.9±1.6 months for partial responders. Patients with partial response had more frequent headaches at baseline compared to the complete responders. Adverse effects were limited to transient local pain at the injection site, and ecchymosis. BTX-A is a safe, effective therapeutic for prophylactic treatment of many cases of migraine.

Toxin Type A (BOTOX) in the Prophylactic Treatment of Migraine

The study was conducted to evaluate the safety and efficacy of pericranial Botox injections as prophylactic treatment of episodic moderate to severe migraine. Subjects with episodic International Headache Society (IHS)-defined migraine and a history of 2 to 8 moderate to severe migraines during a 1-month baseline period were randomized to treatment with 0 U (vehicle), 25 U, or 75 U Botox injected symmetrically into glabellar, frontalis, and temporalis muscles. Daily headache diaries were kept for the baseline period and for 3 months postinjection.

A total of 123 subjects (85% female, mean age 44 years) were enrolled. Between 40 and 42 subjects were randomly assigned to each treatment group. At baseline, the frequency of moderate to severe migraine/month (as classified by the IHS) was 4.44, 4.45, and 3.95 attacks/month in the vehicle, 25 U, and 75 U Botox treatment groups respectively. The 25 U Botox treatment group performed significantly better than vehicle by the following measures: reduction in mean frequency of moderate to severe migraines during months 2 and 3; percent of subjects with a >50% decrease in frequency of any migraines during month 3; percent of subjects with a decrease of >2 in any migraines during month 3; reduction in mean frequency of any migraines during month 3; reduction in maximum migraine severity during months 1 and 2; incidence of subjects with migraine-associated vomiting during month 3; reduction in the number of days in which acute migraine medications were used during month 2; and improvement in subject global assessment at month 2. Botox treatment was well tolerated, with only 75 U Botox associated with significantly better than vehicle in subject global assessment at month 2. Botox treatment was well tolerated, with only 75 U Botox associated with significantly more treatment-related adverse events than vehicle. No serious treatment-related adverse events were reported in any treatment group.

Pericranial injection of 25 U Botox showed significant benefit compared to vehicle in reducing migraine headache frequency, maximal severity, associated vomiting, and number of days using acute medications during the 3 months following injection. Injection of 25 U and 75 U of Botox into pericranial muscles decreased the frequency and severity of migraines and associated vomiting.

Subjects continued to improve through month 3, showing that further improvement can be seen in longer studies. The most consistent and significant improvements were seen with 25 U Botox. The effect of 75 U Botox can have been less pronounced due to fewer headaches at baseline and frequency of adverse events in the group. The above studies leave no question as to the role of upper facial and occipital muscles in relation to the MH.

Surgical Procedures

Transpalpebral Corrugator Supercilii Muscle Resection

Under deep sedation, 1% Xylocaine containing 1:100,000 Epinephrine can be injected in the upper eyelid and lower forehead. An incision, approximately one inch long, can be made in each upper eyelid crease and can be taken through the orbicularis muscle only. In the plane between the skin and orbicularis muscle and orbital septum, the dissection can be continued cephalically until the CSM is exposed. Preserving the supraorbital and supratrochlear nerves, the muscle can be removed as thoroughly as feasible. A small amount of excess fat often protruding on the medial aspect of the upper eyelid can be removed through a small rent in the orbital septum and can be applied to the CSM site to minimize the potential for a depression resultant from removal of the muscle and to create a pliable shield around the nerve branches. The fat graft can be sutured in place using 6-0 Vicryl. The skin can be repaired using 6-0 fast-absorbing catgut. The technique has been developed and is now used routinely internationally. The procedure can be completed bilaterally in 1.5 hours. During the recovery period patients can observe some swelling and/or bruising and numbness in the forehead and orbital region. The patients can resume light activities the next day and heavier activities in about 10 days to 2 weeks, in most incidences.

Temporal Release

Temporal release can be done through an endoscopic technique: After infiltration of the non hair-bearing forehead skin with 1% Lidocaine containing 1:100,000 epinephrine, and the hair-bearing skin with 1:200.000 epinephrine, four port sites can be marked, two on either side in the temple area, each about Ii2-inch in length, and the scalp incisions can be made. After insertion of endoscopic access devices, subperiosteal dissection can be conducted towards the supraorbital rim, lateral orbital rim, zygomatic arch, and malar region. The periosteum and the arcus marginalis can be released over the lateral orbital region and the supraorbital area. The zygomaticotemporal branches of the trigeminal nerve can be transected. Suspension of the fascia can be done using fascial sutures laterally and bone tunneling through the medial incision in the temple area to reposition the soft tissue and reduce the potential for the nerve coaptation. (The technique is routinely performed during aesthetic forehead surgery.) A suction drain can be placed in position and fixed to the skin using 5-0 plain catgut. The incisions can be repaired using a combination of 5-0 Vicryl for the deeper layer and 5-0 plain catgut for the skin. The procedure can generally be completed bilaterally in 1-1.5 hours. Patients can experience some swelling and/or bruising in the temple area or the eyelids. Transient temple paresthesia is expected after surgery and can last up to one year. The patients can resume light activities the next day and normal activities in one week to ten days, in most incidences.

CSM Resection with Temporal Release

The combination of corrugator resection and temporal release is done through an endoscopic approach. The forehead can be infiltrated with 1% Xylocaine containing 1:100,000 Epinephrine in the non-hair bearing area and 1% Xylocaine containing 1:200,000 Epinephrine in the hair bearing sites. Five ports can be designed and the scalp incisions can be made, each Y2-inch in length, one in the midline, and two on either side in the hair bearing temple area. The endoscopic access devices can be inserted. Subperiosteal dissection can be conducted to the supraorbital rim, lateral orbital rim, zygomatic arch, and malar region using an endoscope. The zygomaticotemporal nerve can be transected. The supraorbital nerves and the CSM can then be exposed bilaterally. The glabellar area can also be dissected and the periosteum can be released. The CSM can then be resected as thoroughly as possible preserving the nerves it surrounds. After assuring hemostasis, a piece of fat graft can be applied to the corrugator sites. Suspension can be done using fascial sutures laterally and bone tunneling through the medial incision in the temple area. A suction drain can be placed in position and fixed to the skin using 5-0 plain catgut. The incisions can be repaired using a combination of 5-0 Vicryl and 5-0 plain catgut. The procedure can be completed in 1.5-2 hours. All of the patients can experience transient anesthesia or hypoesthesia. Patients can experience some swelling and/or bruising in the forehead area and periorbital region. Patients can resume light activities the next day and more strenuous activities in about 10 days to 2 weeks, in most incidences.

Occipitalis Muscle Resection

Removal of the occipitalis muscle can be done through a horizontal incision after deep sedation and infiltration of the area with 1% Xylocaine containing 1:100,000 Epinephrine in the hair-bearing skin in the occiput area. A skin flap can be raised and the muscles can be exposed and carefully dissected and removed, freeing the greater occipitalis nerve and its branches. A suction drain can be placed in position and detail repair can be done using a combination of 5-0 Vicryl and 5-0 plain catgut. The bilateral procedure can be completed in 1 hour. Patient can experience some swelling and/or bruising in the occipital area. The subjects can resume light activities the next day and rugged activities in about 10 days to 2 weeks, in most incidences.

Septoplasty/Inferior Turbinectomy

For the septoplasty, a left-sided L-shaped incision can be made on the mucoperichondrium. A flap can be elevated and the septal cartilage can be exposed. The deviated portion of the cartilaginous septum, vomer plate, and perpendicular plate can be removed. If necessary, the nasal spine can be osteotomized and repositioned along with the caudal anterior portion of the septum. Should an enlarged inferior turbinate accompany the septal deviation, a conservative partial turbinectomy can prove necessary to provide sufficient space for the septum to be repositioned. Doyle stents can be placed to help stabilize the septum in the desired position. Bloody nasal drainage can be present for up to one week and the upper front teeth can become numb temporarily. The stents can be removed in 6 to 7 days. The operation can require 45 minutes to 1 hour. Generally the patients can resume light activities the next day and more energetic exercises in one week.

There can be some pain and discomfort associated with anyone of the above procedures, particularly for the first day or two and the patients can receive a prescription for pain medication, as needed. All patients can use their medication should they experience MH.

Surgical Variables

There are three major target areas that can be subjected to injection of Botox for prognostic and trigger point detection purposes. Four total trigger points can be eliminated in a variety of combinations (as follows) based on patient response to the injection of Botox and the results can be statistically analyzed, for example: removal of corrugator supercilii muscle alone; removal of corrugator supercilii muscle and temporal release with transection of the zygomaticotemporal branch of the trigeminal nerve; temporalis release and transection of the zygomaticotemporal branch of the trigeminal nerve alone; removal of corrugator supercilii muscle, release of temporal area with transection of the zygomaticotemporal branch of the trigeminal nerve, and removal of the occipitalis muscle; removal of corrugator supercilii muscle, release of temporal area with transection of the zygomaticotemporal branch of the trigeminal nerve, removal of the occipitalis muscle, and septoplasty with or without partial inferior turbinectomy; temporalis release with transection of the zygomaticotemporal branch of the trigeminal nerve and removal of the occipitalis muscle; temporalis release with transection of the zygomaticotemporal branch of the trigeminal nerve, removal of the occipitalis muscle, and septoplasty with or without partial inferior turbinectomy; removal of the corrugator supercilii muscle and occipitalis muscle; removal of the corrugator supercilii muscle and septoplasty with or without partial inferior turbinectomy; removal of occipitalis muscle alone; removal of occipitalis and septoplasty with or without partial inferior turbinectomy; temporalis release with transection of the zygomaticotemporal branch of the trigeminal nerve with septoplasty with or without partial inferior turbinectomy; septoplasty with or without partial inferior turbinectomy; transection of the zygomaticotemporal branch of the trigeminal nerve with or without partial inferior turbinectomy; removal of the corrugator supercilii muscle, occipitalis muscle, and septoplasty with or without partial inferior turbinectomy; and removal of corrugator supercilii muscle, temporal release with transection of the zygomaticotemporal branch of the trigeminal nerve and septoplasty with or without partial inferior turbinectomy.

### Example 4

Description of Release of Greater Occipital Nerve

With the patient in a sitting position, an incision is designed in the midline upper cervical region approximately 4 cm in length confined to the hair bearing skin. The incision begins approximately 1 cm caudal to the caudal border of skull by palpation. With the patient in the supine position general anesthesia is then induced and a LMA tube is placed to minimize the potential for irritation of the trachea. The patient is then placed in a prone position. The shoulders are lifted with padding.

The neck is then flexed as far as possible within safe limits. A small amount of hair is shaved around the previously designed cephalocaudal incision. The occipital area and the upper cervical region is then prepped and draped. An adhesive drape is used to isolate the hair. The area is then infiltrated with Xylocaine containing 1:200,000 epinephrine. An incision is made through the skin using a 10 blade and taken through the subcutaneous tissues using the quagulation of centers to the midline raphe. At the first level, the incision is shifted slightly away from the midline, and the trapezius fascia is incised. Immediately below the fascia one can identify the semispinalis capitis muscle while the fascia is pulled anteriorly and laterally.

Retracting the combination of a self-retainer and pair of double hooks, the muscle becomes more exposed. With a pair of baby Metzenbaum scissors, the dissection is continued laterally in the subfascial plane. Approximately 1.5 cm from the midline, one almost invariably can identify the trunk of the greater occipital nerve piercing the muscle and reaching the subfascial plane. Using a pair of monion clamps, the dissection is then conducted between the nerve and the muscle fibers in a cephalocaudal direction. The monion is then advanced medially in the submesenteric plane incorporating many muscle fibers. While the monion lifted the muscle, the semispinalis capitis muscle fibers are then transected caudally and cephalically removing approximately 1” of the muscle medial to the nerve.

The procedure is continued to the deeper layers until the nerve is completely uncovered and no fibers are left medial to the nerve. Further dissection can identify a fibrous tunnel deeper to the muscle, which is a release if noted. Next, the nerve is dissected laterally. Any fibrous bands of fascia encasing the greater occipital nerve are released. The dissection again is continued further laterally until the subcutaneous plane is reached. After assurance that the entire extent of the greater occipital nerve is free on one side, the procedure is repeated on the opposite side.

While the midline raphe is retracted, an incision is made through the trapezius fascia on the contralateral side and the semispinalis muscle is exposed and the procedure is completed. Should there be a bifurcation of the greater occipital nerve, which is not uncommon, any muscle existing between the branches is removed. If the greater occipital nerve is found wrapped around the remaining fibers of the muscle, an additional segment of the muscle is removed in order to avoid any undue tension or pressure on the nerve and hemostasis is carefully secured.

Throughout the procedure, the coagulation power of the cautery, set at approximately 35 milliamperes, serves to cut and coagulate the muscle fibers and minimize the potential for a postoperative collection of blood. Having released both greater occipital nerves, a caudally based subcutaneous flap is elevated approximately 2×2 cc. The flap is rotated caudally and sewn to the midline raphe and deeper fascia to prevent continuity of any regeneration muscle and creation of a muscle ring around the nerve. The method further provides a cushion effect around the nerve and minimizes the potential for scar tissue formation. Next, a TLS suction drain is inserted in place. The subcutaneous tissue is then repaired using an inverted 5-0 Vicryl suture while a healthy purchase is made through the midline raphe at the same time. Thus assuring elimination of dead space and proper approximation of the skin to the underlying deeper structures. The subcutaneous tissue is further closed using 5-0 Vicryl and the skin is closed using 5-0 plain catgut interrupted running sutures. Hemostasis is carefully secured throughout the operation.

Description of Release of Zygomaticotemporal Branch of the Trigeminal Nerve and Corrugator Resection

With the patient in the supine position under deep sedation, the face was sterilely prepped and draped. Five radial incisions, each one about 1.5 cm long, were designed with one starting from the midline and the next two placed approximately 7 cm and 10 cm from the midline. The forehead, temple, malar region, and the scalp were injected with Xylocaine containing 1:100,000 epinephrine for non-hair bearing area while 1:200,000 epinephrine was utilized for areas that are covered with hair. The most lateral incision on the right side was made first using a 15 blade. Using a pair of baby Metzenbaum scissors, the incision was then deepened until the deep temporal fascia is exposed.

Using a periosteal elevator then the dissection is conducted medially, laterally, cephalad and caudally to accommodate the Endoscopic Access Device. Next, the periosteal elevator is used to dissect under the second incision, located approximately 7 cm from the midline. The dissection was conducted in the subperiosteal level. The Endoscopic Access Device was then inserted in the incision. The periosteal elevator was then used to raise the periostium posteriorly and cephalically in a blind fashion. An incision was then made in the midline and taken through to the periostium. The periostium was elevated and the Endoscopic Access Device was placed in position. The procedure was repeated on the left side.

Next, under endoscopic visualization, dissection was continued along the lateral orbital rim to the malar arch and the malar region. Both the zygomaticotemporal and some zygomaticofacial branches were transected using coagulation power of the cautery. A similar procedure was done on the opposite side. Next, an incision was made immediately cephalad to the zygomatic arch through the deep temporal fascia exposing the temporal fat deep to the fascia. Fat was harvested from the space and placed in a moist sponge. Next, using a curved periosteal elevator the periostium was elevated, and the periorbit was released laterally and cephalically.

Immediately above the supraorbital rim the supraorbital nerve can be seen. Preserving the nerve integrity, the corrugator muscle was then exposed with teasing effects of the curved periosteal elevator. The muscle fibers were teased from surrounding structures in upward brush strokes with the open jaws of the grasper. When a sufficient bundle was procured, the jaws were closed and the grasper withdrawn. The muscle was then removed as thoroughly as feasible including the procerus and the depressor supercilii muscle. A similar procedure was done on the opposite side. The fat pieces harvested from the temporal fossa were delivered and distributed across the forehead evenly in the muscle sites as well as the glabella region. The Endoscopic Access Devices were then removed.

Next, a 3-0 PDS suture was passed through the superficial and intermediate temporal fascia at the caudal portion of the most lateral incision, starting from the deeper portion, which was done after placing a single hook on ether side of the incision caudally. Then the skin hooks were replaced along the cephalic margins and the tissues were pulled cephalically and minimally posteriorly. The suture was then passed through the deep temporal fascia and tied.

A drain was placed in position and passed from one hole to the other extending from the right to the left side of the scalp in the subperiosteal plane. The drain was anchored in position and a detail repair was done using a combination of 5-0 Vicryl and 5-0 plain catgut interrupted sutures. It was essential to close the deeper layers in order to avoid a sunken appearance.

Throughout the application, various publications, including United States patents, are referenced by author and year, and patents, by number. Full citations for the publications are listed below. The disclosures of the publications and patents in their entireties are hereby incorporated by reference into the application in order to more fully describe the state of the art to which the invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

TABLE I
Entire Patient Population

| | | | | Before Treatment | | | After Botox Injection | | | After Surgery | | | |
|---|---|---|---|---|---|---|---|---|---|---|---|---|---|
| | | | | Corrugator | Migraine | Migraine | Migraine | Migraine | | Migraine | Migraine | Days | Sur- |
| | | | | Supercilii | Headache | Headache | Headache | Headache | Botox | Headache | Headache | of | gical |
| Pa- | Age | | Aura/ | Muscle | Frequency | Intensity | Frequency | Intensity | Out- | Frequency | Intensity | Follow- | Out- |
| tient | (years) | Gender | Nonaura | Hypertrophy | per Month | per Month | per Month | per Month | come | per Month | per Month | Up | come |
| 1 | 47 | F | A | 4 | 6 | 9 | 2 | 8 | SD | 1.8 | 7.2 | 313 | SD |
| 2 | 40 | F | A/N | 5 | 8 | 9 | 0 | 0 | E | 0.2 | 6.0 | 320 | SD |
| 3 | 49 | M | N | 5 | 8 | 9 | 2 | 6 | SD | 0.7 | 9.5 | 277 | SD |
| 4 | 40 | F | A | 5 | 3 | 10 | 0 | 0 | E | 0 | 0 | 382 | E |
| 5 | 42 | F | N | 5 | 4 | 9 | 0 | 0 | E | | | | |
| 6 | 35 | F | N | 5 | 8 | 9 | 12 | 7 | NR | | | | |
| 7 | 38 | F | A | 5 | 5 | 10 | 2 | 9 | SD | | | | |
| 8 | 44 | M | A | 4 | 4 | 10 | 0 | 0 | E | 0 | 0 | 461 | E |
| 9 | 47 | M | A | 4 | 5 | 8 | 2 | 7 | SD | 3.2 | 6.8 | 347 | SD |
| 10 | 53 | F | A | 5 | 3 | 10 | 0 | 0 | E | 0 | 0 | 361 | E |
| 11 | 41 | F | N | 5 | 6 | 7 | 0 | 0 | E | 6.0 | 6.7 | 311 | NR |
| 12 | 49 | F | A | 4 | 6 | 8 | 0 | 0 | E | 0 | 0 | 494 | E |
| 13 | 48 | F | A | 5 | 4 | 8 | 0 | 0 | E | 0 | 0 | 326 | E |
| 14 | 63 | M | N | 4 | 6 | 9 | 15 | 9 | NR | | | | |
| 15 | 58 | M | N | 5 | 9 | 7 | 2 | 8 | SD | 0 | 0 | 340 | E |
| 16 | 53 | F | A | 4 | 3 | 9 | 0 | 0 | E | 0 | 0 | 312 | E |
| 17 | 47 | F | N | 5 | 4 | 8 | 1 | 4 | SD | 0.1 | 9 | 347 | SD |
| 18 | 34 | F | A | | 5 | 9 | 0 | 0 | E | 0.7 | 6.4 | 404 | SD |
| 19 | 53 | F | N | 5 | 4 | 9 | 5 | 7 | NR | | | | |
| 20 | 45 | F | N | 5 | 5 | 8 | 5 | 4 | SD | 1.5 | 5.7 | 340 | SD |
| 21 | 30 | F | A | 5 | 3 | 8 | 0 | 0 | E | 0 | 0 | 222 | E |
| 22 | 29 | F | A | 4 | 5 | 9 | 0 | 0 | E | 0.8 | 8 | 362 | SD |
| 23 | 49 | F | N | 5 | 9 | 10 | 0 | 0 | E | 1.5 | 9.7 | 306 | SD |
| 24 | 24 | F | A | 5 | 5 | 9 | 0 | 0 | E | 0 | 0 | 322 | E |
| 25 | 63 | F | N | 4 | 19 | 7 | 10 | 7 | NR | | | | |
| 26 | 62 | F | A | 5 | 8 | 9 | 11 | 9 | NR | | | | |
| 27 | 44 | F | A | 5 | 3 | 10 | 0 | 0 | E | 0.3 | 8.5 | 349 | SD |
| 28 | 38 | F | N | 4 | 8 | 10 | 1 | 3 | SD | 0 | 0 | 341 | E |
| 29 | 38 | F | N | 5 | 3 | 10 | 0 | 0 | E | 0.2 | 7.0 | 389 | SD |
| Av- | 44.9 | F 24 | A 14 | 4.7 | 5.9 | 8.7 | 2.4 | 3.0 | E 16 | 0.9 | 4.1 | 347 | E 10 |
| erage | | M 5 | N 14 | | | | | | SD 8 | | | | SD |
| | | | | | | | | | | | | | 11 |
| | | | | | | | | | NR 5 | | | | NR 1 |

E, elimination;
SD, significant decrease;
NR, no response;
A, aura;
N, nonaura;
M, male;
F, female.

TABLE II
Results of Botox Injection and Surgery

| | | After Botox | |
|---|---|---|---|
| | Before | Injection/ | p |
| | Treatment | Surgery | Value* |
| Group with elimination of MH | | | |
| after Botox injection (n = 16) | | | |
| Average MH frequency | 4.6 | 0 | <0.001 |
| Average MH intensity | 9.1 | 0 | <0.001 |
| Group with significant decrease in | | | |
| MH after Botox injection (n = 8) | | | |
| Average MH frequency | 6.4 | 2.1 | <0.04 |
| Average MH intensity | 8.6 | 6.1 | <0.04 |
| Group with no response to injection | | | |
| of Botox (n = 5) | | | |
| Average MH frequency | 9.0 | 10.6 | NS |
| Average MH intensity | 8.6 | 7.8 | NS |
| Group with elimination of MH | | | |
| after surgery, mean follow-up: | | | |
| 356 days (n = 10) | | | |
| Average MH frequency | 4.8 | 0 | 0.01 |
| Average MH intensity | 8.9 | 0 | 0.01 |
| Group with significant decrease in | | | |
| MH after surgery, mean follow-up: | | | |
| 341 days (n = 11) | | | |
| Average MH frequency | 5.6 | 1.0 | 0.004 |
| Average MH intensity | 9.0 | 7.5 | 0.04 |
| Patient with no response to surgery, | | | |
| follow-up: 311 days (n = 1) | | | |
| Average MH frequency | 6.0 | 6.0 | — |
| Average MH intensity | 7.0 | 6.7 | — |

MH, migraine headache;
NS, not significant.
*Using Wilcoxon signed rank test.

TABLE 3
MIGRAINE STUDY DESCRIPTIVES
Descriptive Statistics

| | N | Minimum | Maximum | Mean | Std. Deviation |
|---|---|---|---|---|---|
| MH Number | 21 | 3 | 9 | 5.33 | 2.03 |
| Valid N | | | | | |
| (listwise) | | | | | |

FOLLOW-UP DAYS
Descriptive Statistics

| | N | Minimum | Maximum | Mean | |
|---|---|---|---|---|---|
| | Statistic | Statistic | Statistic | Statistic | Std. Error |
| DAYSFU | 21 | 56 | 273 | 131.57 | 11.21 |
| Valid N | 21 | | | | |
| (listwise) | | | | | |

MIGRAINE SEVERITY
Descriptive Statistics

| | N | Minimum | Maximum | Mean | Std. Deviation |
|---|---|---|---|---|---|
| | Statistic | Statistic | Statistic | Statistic | Std. Error |
| SEVERITY | 21 | 7 | 10 | 8.9 | 0.22 |
| Valid N | 21 | | | | |
| (listwise) | | | | | |

What is claimed is:

1. A method of treating pain caused by a nerve being compressed by a muscle, the method including the step of inactivating the muscle causing the nerve compression.

2. The method according to claim 1, wherein said inactivating step includes removing the muscle.

3. The method according to claim 1, wherein said removing step includes operating to remove the muscle.

4. The method according to claim 1, wherein inactivating step includes permanently paralyzing the muscle.

5. The method according to claim 4, wherein said paralyzing step includes administering a compound for paralyzing the muscle.

6. A treatment for eliminating pain associated with nerve compression, said treatment comprising muscle inactivating means for inactivating the muscle and relieving the nerve compression.

7. The treatment according to claim 6, wherein said muscle inactivating means is a compound for paralyzing the muscle.

8. The treatment according to claim 6, wherein said muscle paralyzing means is physical apparatus for paralyzing the muscle permanently.

9. An algorithm for determining the location of pain causing nerve compression.
